(19) United States
(12) Plant Patent Application Publication
(10) Pub. No.: US 2014/0215672 P1
(43) Pub. Date: Jul. 31, 2014

(54) Stromanthe 'ROTOLANTE10'

(50) ;

(71) Applicant: SILVER KROME GARDENS, INC., Homestead, FL (US)

(72) Inventors: Denis Rotolante, Homestead, FL (US); Charles W. McDaniel, West Palm Beach, FL (US)

(73) Assignee: SILVER KROME GARDENS, INC., Homestead, FL (US)

(21) Appl. No.: 13/815,093

(22) Filed: Jan. 30, 2013

(51) Int. Cl. A01H 5/00

(52) U.S. Cl. PLT/373

(57) ABSTRACT

A new and distinct variety of stromanthe plant named ‘ROTOLANTE10’ particularly distinguished by its bright, broad leaf variegation, compact growth habit, short petioles, and tolerance to a broad range of light regimes, is disclosed.

### GENUS AND SPECIES

Stromanthe stromanthoides

### VARIETY DENOMINATION

‘ROTOLANTE10’

### BACKGROUND OF THE NEW PLANT

The present invention relates to a new and distinct stromanthe variety designated ‘ROTOLANTE10’ and botanically known as Stromanthe stromanthoides. The new stromanthe was discovered and selected by the inventors from a naturally occurring, single plant mutation of stromanthe within a population of stromanthe plants in a controlled greenhouse environment in Homestead, Fla. This single plant was selected for further evaluation and for asexual propagation in Homestead, Fla. based on its bright leaf variegation pattern, compactness of growth and ease of growth.

The new variety was first propagated via vegetative cuttings in the summer of 2002 in Homestead, Fla. and has been asexually reproduced repeatedly by vegetative cuttings and tissue culture in Homestead, Fla. for 12 generations. ‘ROTOLANTE10’ has been found to retain its distinctive characteristics through successive asexual propagations by vegetative cuttings and tissue culture.

Plant Breeder's Rights for this variety have not been applied for. ‘ROTOLANTE10’ has not been made publicly available more than one year prior to filing of this application

### SUMMARY OF THE INVENTION

The following are the most outstanding and distinguishing characteristics of this new cultivar when grown under normal horticultural practices in Homestead, Fla.

1. Bright, broad leaf variegation;

2. Compact growth habit with short petioles; and

3. Tolerance to a broad range of light regimes.

### DESCRIPTION OF PHOTOGRAPH

This new stromanthe plant is illustrated by the accompanying photographs. The colors shown are as true as can be reasonably obtained by conventional photographic procedures. The photographs are of 1-year old plant propagated by vegetative cuttings from rhizomes.

FIG. 1 shows a whole plant.

FIG. 2 shows a close up the mature foliage.

FIG. 3 shows a close up of the mature foliage and a mature bract.

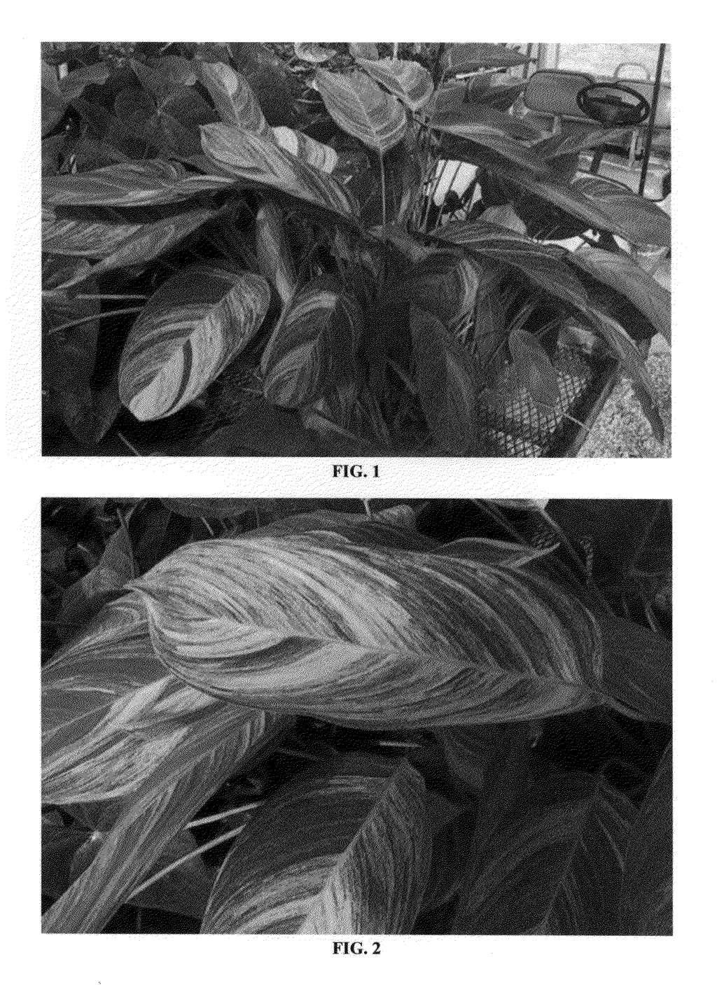

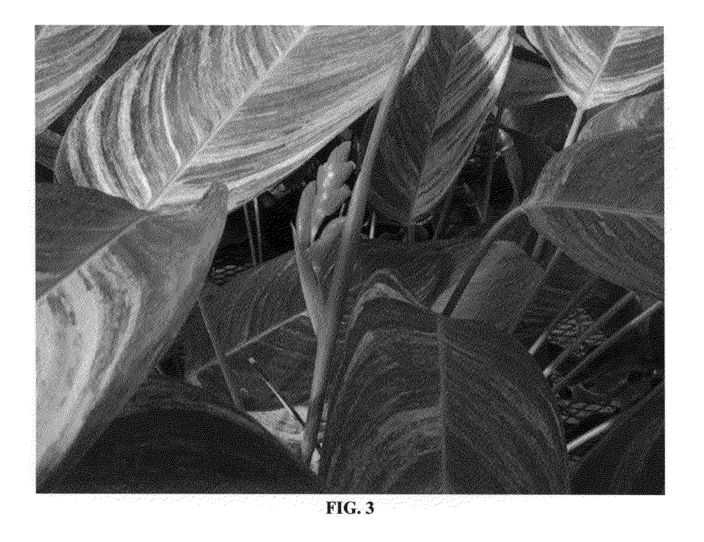

### DESCRIPTION OF THE NEW CULTIVAR

The following detailed description sets forth the distinctive characteristics of ‘ROTOLANTE10’. The data which define these characteristics were collected from asexual reproductions carried out in Homestead, Fla. The plant history was taken in the summer of 2012 on one-year old plants grown in 15 cm pots. The plants were grown in a greenhouse covered in six mill polyethylene film and 73% shade cloth. Color readings were taken under natural light. Color references are primarily to the R.H.S. Colour Chart of The Royal Horticultural Society of London (R.H.S.), Third Edition (1995).

### DETAILED BOTANICAL DESCRIPTION OF THE NEW PLANT

Classification:
   Family.—Marantaceae.
   Botanical name.—Stromanthe stromanthoides.
   Common name.—Stromanthe.
Growth:
   General.—Tropical potted plant; plant is suitable for use in shaded tropical and frost-free landscapes.
   Container size.—4 liter (15.0 cm diameter) and 12 liter (25.5 cm diameter).
Plant description:
   Form.—Erect.
   Life cycle.—Herbaceous perennial herb.
   Habit.—Upright and branching from the rhizomes at the base of the plant.
   Vigor.—Medium to fast growth rate.
   Height (from top of soil).—35.0 cm.
   Width (horizontal plant diameter).—40.0 cm.
   Time to produce a finished plant.—2 liter container (6 inch or 15 cm in diameter): 6 months 12 liter container (10 inch or 25 cm in diameter): 11 to 12 months.
   Time to initiate and develop roots from unrooted cuttings.—2 to 4 weeks.
   Root description.—Primary adventitious roots dominant from the rhizome branching to secondary rootlets.
   Propagation method.—Tissue culture and vegetative cuttings.
Branches:
   Number.—4 to 6.
   Length.—Variable depending upon age.
   Lateral branch diameter.—1.5 cm.
Stems (rhizomes):
   Description.—Cylindrical, round in traverse section.
   Aspect.—Horizontal, underground rhizome becoming upturned, vertical at tip.
   Strength.—Thick, tough and fibrous.
   Internode length.—1.0 cm to 2.0 cm.
   Color.—RHS 158D.
Leaves:
   Arrangement.—Alternate, closely spaced in vertical ranks, rosette, with 1 to 3 per lateral branch depending on age of the branch.
   Shape.—Simple, elliptical.
   Apex.—Obtuse with acuminate tip.
   Base.—Asymmetrical, obtuse.
   Margin.—Entire; very slightly concaving along the margins.
   Length.—15.0 cm to 28.0 cm.
   Width.—6.0 cm to 11.0 cm.
   Color (both immature and mature leaves).—Upper surface: RHS 158D to RHS 142A Lower surface: RHS 158D to RHS 142D.
   Venation color (both upper and lower surfaces).—RHS 158D.
   Variegation (both immature and mature leaves).—Upper surface: RHS 158D to RHS 142A Lower surface: RHS 158D to RHS 142D.
   Petiole.—Aspect: Upright Length: 15.0 cm to 30.0 cm Diameter: 0.2 cm to 0.4 cm Texture: Smooth Color: RHS 158D.
   Geniculum.—Aspect: The orientation of the leaf to the petiole is variable, as the geniculum bends; during the night and early morning the geniculum is straight and the leaf is held nearly vertical above the foliage; during the day, the geniculum is bent and the leaf curves outward Length: 1.0 cm to 2.0 cm Diameter: 0.3 cm to 0.5 cm Color: RHS 158D Texture: Smooth.
Leaf sheath:
   Length.—5.0 cm to 10.0 cm.
   Diameter.—0.5 cm.
   Apex.—Acute.
   Texture.—Smooth.
   Color.—RHS 158D.
Inflorescence:
   Arrangement.—Sparingly arising from the axils of the leaves.
   Time of year observed.—Spring and summer.
   Quantity of inflorescence per plant.—1 to 2.
   Fragrance.—Absent.
   Lastingness of inflorescence.—21 days.
   Peduncle.—Length: 15.0 cm to 25.0 cm Diameter: 0.2 cm to 0.4 cm Angle: Upright to 15 degrees from center Strength: Medium strong Texture: Smooth Color: RHS 158D.
   Bracts.—Aspect: Overlapping Shape: Elliptical, concave Apex: Obtuse Base: Obtuse Margin: Entire Texture: Smooth Length: 1.5 cm to 2.0 cm Width: 1.0 cm to 1.5 cm Color: Immature (both upper and lower surface): RHS 24A Mature (both upper and lower surface): RHS 24A Flowers: Flower development has not been observed on plants of the new stromanthe.
Fruit and seed set: Has not been observed
Disease and insect resistance: Good disease resistance with some possibility of mites
Hardiness zones: USDA 10 to 11
Drought tolerance: Moderate

### COMPARISON WITH COMMERCIAL CULTIVARS

‘ROTOLANTE10’ can be distinguished from the commercial cultivar stromanthe plant named ‘Valmic’ (U.S. Plant Pat. No. 15,475) in that ‘ROTOLANTE10’ has orange bracts with no flowers and green coloration on the lower leaf surface, whereas ‘Valmic’ has red bracts with small white flowers and red coloration on the lower leaf surface. Additionally, ‘ROTOLANTE10’ has broader leaves, longer petioles and a more open appearance than ‘Valmic’.

We claim:

1. A new and distinct variety of stromanthe plant named ‘ROTOLANTE10’ as shown and described herein.
